(19) United States
(12) Patent Application Publication
(10) Pub. No.: US 2026/0215802 A1
(43) Pub. Date: Jul. 30, 2026

(54) MEDICAL SYSTEM AND METHOD OF USE

(71) Applicant: John H. SHADDUCK, Menlo Park, CA (US)

(72) Inventor: John H. SHADDUCK, Menlo Park, CA (US)

(21) Appl. No.: 19/431,446

(22) Filed: Dec. 23, 2025

Related U.S. Application Data
(60) Provisional application No. 63/740,198, filed on Dec. 30, 2024.
(60) Provisional application No. 63/745,706, filed on Jan. 15, 2025.

(51) Int. Cl. A61B 17/3203; A61B 1/00; A61B 1/05; A61B 1/307; A61B 17/00; A61B 17/32; A61B 34/10; A61B 34/30

(52) U.S. Cl. CPC A61B 17/3203; A61B 1/00006; A61B 1/000094; A61B 1/05; A61B 1/307; A61B 17/320016; A61B 34/10; A61B 34/30; A61B 2017/00022; A61B 2017/00137; A61B 2017/00402; A61B 2017/00539; A61B 2017/00743; A61B 2034/107; A61B 2034/303

(57) ABSTRACT

Medical robotics and surgical systems designed to treat benign prostatic hyperplasia that use autonomous, semi-autonomous, and AI-assisted systems that including mapping for volumetric resection followed by automated surgical resection, extraction, and cauterization of the treated prostate.

### RELATED APPLICATION INFORMATION

The present application is a non-provisional of U.S. Provisional No. 63/740,198 filed Dec. 30, 2024 and U.S. Provisional No. 63/745,706, filed Jan. 15, 2025, the entirety of both of which are incorporated by reference.

### FIELD OF THE INVENTION

The present invention relates to the field of medical robotics and, more specifically, to surgical systems designed to treat benign prostatic hyperplasia that use autonomous, semi-autonomous, and AI-assisted systems that including mapping for volumetric resection followed by automated surgical resection, extraction, and cauterization of the treated prostate.

### BACKGROUND OF THE INVENTION

Benign prostatic hyperplasia (BPH) is a prevalent condition among elderly men with increasing prevalence as men age and affects upwards of 60% of men by the age of 65. BPH consists of the progressive benign enlargement of the prostate gland, primarily attributable to unregulated hyperplastic growth in the epithelial and fibromuscular tissues of the transition zone and periurethral area of a human prostate, resulting that restricts flow from the bladder through the prostatic urethra.

Surgical interventions are a viable option in treating BPH with transurethral resection of prostate (TURP), historically regarded as a gold standard for small to moderately sized prostates. In a TURP procedure, a substantial volume of the patient's prostate gland is resected with an RF electrosurgical loop (a resectoscope), and the extraction of tissue reduces pressure on the prostatic urethra. The electrosurgical devices used in TURP procedures have the advantage of cauterizing the surface of the resected tissue, so post-treatment bleeding is not an issue. Other minimally invasive surgical treatments and implants for treating BPH are known in the prior art. However, volumetric resection, as in TURP procedure, provides the most immediate relief BPH symptoms. A significant disadvantage of a TURP resection is that the procedure is skill-dependent and requires 60 to 90 minutes in the operating room, making it a very costly treatment.

Water jet cutting in surgical applications. Water jet cutting technology has gained significant traction in many industries, including surgical applications. High-pressure water jet systems have demonstrated efficacy in surgical procedures by offering precise and controlled tissue cutting and obliteration, for example, in orthopedic procedures to precisely cut cartilage or herniated disc material.

Surgical water jet systems typically use high operating pressures, and to control the limits of the cutting effect, the high-pressure water jet is directed towards a “backstop” that comprises a jet evacuation channel. Such surgical water jet devices are designed for precise, rapid removal of small volumes of tissue, often in confined spaces. The water jet carries kinetic energy that is focused on a narrow target area between a jet orifice aimed at the backstop evacuation channel, allowing for controlling the boundaries of the cutting zone and avoiding the risk of collateral damage to tissue.

FIGS. 1A and 1B Illustrate such prior working ends 10 and 12, where a surgical device has an elongate shaft with a working end configured with a high-pressure water jet that is directed towards a jet evacuation channel. FIG. 1A illustrates a water jet that propagates transverse to the axis of the shaft. FIG. 1B illustrates a water jet that propagates in alignment with the axis of the shaft from the jet orifice toward the backstop evacuation channel. FIGS. 1A and 1B illustrate devices commercialized by Hydrocision, Inc. and are used in spine discectomies and similar procedures. (FIGS. 1A and 1B are copied from Hydrocision's U.S. Pat. No. 6,375,635 at FIGS. 5a and 5b with the reference numbers removed for clarity).

The configurations of FIGS. 1A and B are optimized for precise, rapid removal of small volumes of tissue, such as herniated disc material. By confining the jet's energy between the jet orifice and the backstop evacuation channel, these water jet devices achieve highly localized tissue removal. However, these devices are not optimized for the volumetric removal of large tissue volumes, as required in BPH treatment. Further, such water jet devices are designed for resecting tissues that are not highly vascularized, such as disc tissue, as such water jet devices provide no mechanism for cauterizing tissue.

Directly aiming a high-pressure water jet at soft tissue without a “backstop,” as described above, presents significant challenges for predictable depth of cutting. A primary issue lies in the unpredictable nature of the jet-tissue interaction. Factors such as tissue heterogeneity (varying density and stiffness), variations in jet velocity and angle, and the dynamic nature of the cutting process make it difficult to achieve a predictable depth of tissue penetration when using a high-pressure water jet. The high kinetic energy of such a water jet also can lead to unpredictable tissue displacement, deformation, and fragmentation, making it challenging to achieve a consistent and controlled resection with high pressures.

More recently, low-pressure variations of surgical water jet devices have been developed using a “backstop” feature. Such low-pressure devices selectively cut softer, less dense tissue while sparing denser structures due to differences in tissue elasticity and strength. For example, in the liver, a low-pressure water jet skeletonizes tissues in dissecting liver parenchyma while sparing major blood vessels and bile ducts due to their differing mechanical properties. Similarly, in pancreatic resections, a water jet can separate pancreatic tissue from vital ducts and vessels. Unlike other resection tools (RF, laser, etc.), a water jet does not generate significant heat, thus preserving blood vessels and ducts

A low-pressure water jet system has been introduced for volumetric tissue removal to treat BPH, as disclosed in exemplary U.S. Pat. No. 9,364,250 owned by Procept Biorobotics. This system does not use a water jet directed at a “backstop” for controlling cutting depth. This variation of water jet system also robotically moves the water jet in a mapped path in the prostate. This system creates a prostate map using ultrasound data received from a trans-rectal ultrasound system (TRUS) and then maps the cutting zone based on the ultrasound prostate map. This system's lack of a backstop to control cutting depth and use of an ultrasound mapping mechanism is outlined below.

FIGS. 2A and 2B Illustrate low-pressure water jet devices 14 and 16 (FIGS. 2A and 2B are copied from Procept-owned U.S. Pat. No. 9,364,250 at FIGS. 4 and 5 with the reference numbers removed for clarity). The device of FIGS. 2A-2B comprises a shaft adapted for trans-urethral introduction with a water jet that is pointed radially outward from the shaft towards prostate tissue instead of towards a backstop channel as in the surgical water jet devices of FIGS. 1A-1B. In FIGS. 2A-2B, it can be seen that the low-pressure water jet device is moved axially and rotationally to cut tissue. Since the water jet is directed radially outward from the shaft with no backstop, substantially low pressures are required compared to variations of FIG. 1A and 1B. At the lower pressure levels, the water jets often skeletonize tissue as the soft prostate glandular tissue is disintegrated, but larger, more elastic blood vessels remain intact.

In such a prostate resection, the low-pressure water jet inevitably cuts smaller blood vessels, and the incidence of bleeding complications is very common. There remains a high degree of uncertainty regarding how bleeding should be managed during a low-pressure water jet procedure, but a combination of both electrocautery with a resectoscope and the use of traction devices has been reported to yield the best results. However, using an RF resectoscope adds significant unneeded costs to a low-pressure water jet procedure, as well as adding 15 to 30 minutes to the procedure time. Similarly, new traction devices add to the cost of disposable devices needed for the procedure and add time to the procedure.

The use of a TRUS system to create a map of the prostate is known in the prior art, and is time-consuming and may not be completely accurate for programming an automated cutting device. The steps of using a TRUS system include lubrication and insertion of the TRUS probe (2 minutes), imaging and measurements with a caliper (5-10 minutes), scanning the prostate in different planes to obtain measurements of length, width, and height (5-10 minutes), calculating volume and estimating weight and recording the measurements (5-10 minutes). These steps typically would require 15 to 20 minutes and could be longer. Further, the accuracy of a prostate map created from a TRUS system can have a variance from the actual prostate dimensions, and the extent of nay variance depends on several factors. The experience and skill of the clinician performing the ultrasound can significantly impact accuracy. Higher-resolution TRUS systems with the best ultrasound transducers can provide more accurate measurements. Further, the shape and consistency of the prostate, patient movement, rectal gas, and/or the presence of calcifications can all affect image quality and the accuracy of prostate mapping.

A BPH procedure, as described above, using a TRUS mapping system and a robotically controlled low-pressure water jet requires about 60 minutes in the operating room. The low-pressure water jet is only actuated for 3 to 5 minutes in such a procedure to robotically move the jet axially and rotationally. The additional approximately 55 minutes of operating room time is needed for (i) mapping the prostate to determine the profile of the resection and (ii) using electrocautery devices and/or traction devices to stop bleeding in the resected cavity in the patient's prostate.

A need exists for a water jet cutting system that allows for (i) rapid, accurate mapping of the prostate, (ii) very rapid, controlled tissue resection and extraction of tissue debris, and (iii) rapid and effective cauterization of the surface of the resected cavity in the patient's prostate. The present invention aims to address this need by introducing an accurate mapping system, a liquid water jet cutting system that contemporaneously provides effective cauterization of the resected cavity in the prostate.

### SUMMARY OF THE INVENTION

The disclosed invention comprises a multi-component system that uses automated and semi-automated devices for treatment of BPH including mapping of a patient's prostate followed by volumetric resection of prostate tissue and cauterization of the resected cavity in the prostate.

Prostate mapping. In a first aspect of the invention, a mapping tool is introduced through a trans-urethral introducer positioned within the prostate. This tool includes at least one elongated needle with a sharp or semi-sharp tip, designed for motor-driven penetration into prostate tissue with minimal resistance to ensure precise needle placement. Each needle carries at least one dielectric sensor at its needle tip. The dielectric sensors comprise a micro-electrode arrangement that sends electrical signals to a controller, which measures changes in capacitance and other dielectric properties, which vary according to the electrical characteristics of the tissue. For example, the tissue near the prostate capsule shows significantly different capacitance values compared to the inner zones of glandular tissue not near the capsule. The controller analyzes capacitance and, optionally, impedance and phase shift to identify and map the prostate capsule.

For example, tissues consist of cells with membranes surrounded by extracellular fluids, creating two separate electrically conductive compartments: the intracellular and extracellular media. The thin, semi-permeable lipid bilayer of cell membranes, due to its insulating properties, has high capacitance, contributing to capacitive reactance. Prostate capsular tissue differs from inner prostate glandular tissue due to variations in water content, ion concentration, and dielectric properties, all of which affect capacitance. Tissues with higher water content, like muscle, exhibit higher capacitance than fatty tissues. Impedance is affected by the tissue's conductivity and resistivity. Different tissues have distinct electrical conductivities because of variations in ion concentration and cell structure. By analyzing these parameters—capacitance, impedance, and phase shift—software algorithms in the controller can distinguish capsular from glandular tissues.

The controller is configured with software algorithms that analyze the capacitance and other dielectric data to locate capsular tissue, in part based on comparison with libraries of known dielectric values for tissue types. The robotic system is automated and controlled by a controller to manage the stepper motor insertion of the sensing needles and signals from the stepper motors, and deployed needle shape allows for precise mapping of the capsular tissue relative to the stabilized position of the mapping tool shaft. The controller then algorithmically distinguishes between capsular tissue and inner glandular prostate tissue and generates a real-time map of the capsular tissue, often visualized in 3D or rotatable images on a display. This map is then used to program a resecting device tool for the volumetric resection and extraction of tissue only within the mapped boundaries. These algorithms improve over time through machine learning for increased accuracy.

Additionally, the mapping function can be integrated with other existing surgical navigation systems or displays, providing visual guides for tissue resection, including interfacing with imaging systems such ultrasound, fluoroscopy, or MRI for additional visual guidance.

Resection of prostate tissue. In a second aspect of the invention, a jittering or oscillating liquid water jet stream is provided for the volumetric resection and extraction of prostate tissue that uses piezoelectric actuation technology to enhance control of cutting depth. The system incorporates a stacked piezoelectric actuator that provides controlled actuator movements at frequencies up to 20 Hz, which provides selected jitter in the water liquid water jet stream. In a variation, the actuation mechanism is enhanced by a parallelogram-type flexure amplifier, designed to amplify the minimal displacement of the piezoelectric stack (typically of less than 100 microns) to achieve a stroke between 1 to 4 mm.

In conventional water jet cutting of soft tissue, the mechanisms by which a high-pressure water jet applies its kinetic energy to perform cutting, disintegration, or obliteration of tissue involved several physical principles. The liquid water is pressurized to an extremely high level, converting potential energy into kinetic energy as it exits through a nozzle, resulting in a high-velocity water stream. As the liquid jet stream strikes tissue, the stream transfers its kinetic energy through direct impact, causing localized stress and strain leading to mechanical failure at the cellular level. The liquid jet stream's high momentum disrupts cellular bonds, effectively cutting or disintegrating tissue by overcoming the cohesive forces within the tissue matrix.

In terms of operating characteristics of the present invention, the jittering liquid water jet stream introduces transverse shear waves into targeted tissue, which enhances tissue disintegration or cutting—instead of only kinetic energy provided by direct jet stream impact. As the liquid jet stream strikes tissue and jitters, the stream imparts lateral forces, creating shear waves where the displacement of engaged tissue is transverse to the direction or vector of the liquid jet steam propagation. Transverse shear waves enhance tissue disintegration by introducing transverse forces, providing tissue separation through shearing forces rather than only by direct impact or cavitation in the stream's propagation vector. Do to the high velocity of the liquid stream, very high-speed jitter of the jet is required, which is achieved with the stacked, amplified piezoelectric actuation of the jetting orifice, which is typically from 5 Hz to 20 Hz wit a jitter stroke of 0.25 mm to 2.5 mm. Thus, the jet stream's kinetic energy is applied to tissue over the length of the jitter stroke, allowing tissue to relax along the non-impacted portion of the stroke rather than being deformed at a single point of jet impact.

The system thus has a controller that can modulate four operational parameters for controlling the cutting depth, which includes modulating the pressure of the liquid water jet stream, the jitter rate of the piezoelectric actuator, the jitter stroke of the piezoelectric actuator and the overall speed of axial and rotational movement of the jetting shaft and jetting orifice.

The result is liquid water jet with a controller and piezoelectric actuator system capable of fine-tuned motion control of a jetting shaft and jetting orifice that propagates the liquid water jet stream. The design creates what could be termed a “backstop” effect, where the controlled motion of the jetting shaft and jetting orifice can affect the depth the liquid water jet stream's penetration into soft tissue and the kinetic energy applied to disintegrate any tissue or selectively cut tissue based on tissue types.

Cauterization of resected cavity in a prostate. In a third aspect of the invention, the single resection-cauterization device further includes a system and method for cauterization of surfaces of a resected cavity in the prostate. In a variation, the system also is supported by artificial intelligence (AI) and/or machine learning to ensure cauterization is provided during the resection step of the method. The cauterizing system provides a water vapor jet stream exiting a vapor jet orifice in the jetting shaft, wherein the water vapor jet stream undergoes a vapor-to-liquid phase transition in the treatment site to release and apply up to 540 cal/gm of energy in the phase change. The phase change instantly thermally cauterizes tissue in the interface of the vapor-to-liquid phase change. The use of phase change energy released from water vapor to thermally ablate, cauterize or modify tissue is disclosed by the author in U.S. Pat. No. 7,674,259; 11,413,086; 11,207,118; 8,911,430; 8,721,632; 11,129,664; 9,615,875; 10,675,079; 8,579,888; 8,574,226; 8,579,893; 10,595,925; 8,900,223; 8,758,341; 11,284,931; 8,579,892; 11,179,187; 10,548,653; 9,204,889; 11,457,969; 10,499,973; 7,892,229; 9,468,487; 10,524,847; 9,433,457; 9,113,944; 8,313,485; 11,478,291; 9,907,599; 11,141,210; 7,549,987; 8,016,823; 8,444,636; 11,284,932; 9,924,992; 11,672,584, 9,161,801; 9,943,353; 8,858,549; 6,669,694; 8,187,269; 6,911,028 and 6,508,816.

The resection-cauterization device of the invention is further configured to deliver a vapor jet stream from the working of the device, which can cauterize or coagulate prostate tissue very rapidly. The cauterization component comprises a vapor generator device typically carried in a hub of the resecting-cauterization device. The vapor jet can deliver a water vapor that releases 540 cal/gm of energy from the vapor-to-liquid phase change to prostate before resection, during resection, or after resection. To cauterize surfaces of resected prostate tissue, calculations indicate that the total vapor jet delivery interval when delivering energy at 50 cal/sec to 100 cal/sec will be less than 120 seconds, and often less than 60 seconds.

In another aspect of the invention, the automated resection can be assisted with artificial intelligence (AI) and/or machine learning wherein algorithms in the controller are adapted to monitor video imaging from the device's image sensor in real time to identify treatment site parameters and in response thereto can automatically modulate or terminate operation of the liquid jets, pressure of each jet stream, spacing of jet streams, operation of the vapor jet and cal/sec delivered, movement of the resecting assembly, operation of negative pressure source, or adjustment of the robotic arm.

The site parameters that the AI/machine learning algorithm monitors are, at least: image observable colors indicating bleeding, observable bubbles in images that indicate cavitation, observable collapse of side walls of the resection cavity, observable tissue debris that indicates sub-optimal cutting, color of tissue indicating cauterization or coagulation, tissue features indicating prostate tissue types, and identification of verumontanum, ducts and the like in the prostate.

In summary, the total time to deploy and use the mapping tool of the invention to map a patient's prostate and to select a resection profile is expected to take approximately 5 to 10 minutes. The total time interval for robotically resecting and cauterizing prostate tissue with the dual water jet streams is expected to take 5 minutes. Thus, the entire procedure time using the present invention for volumetric reduction and cauterization in a BPH procedure is expected to be in the range of 15 minutes which is much faster than other commercial resection and cauterization methods. It is safer than other procedures since cauterization with a vapor phase change is fast and effective.

### BRIEF DESCRIPTION OF THE DRAWINGS

FIG. 1A is a prior art water jet resection device that directs a high-pressure water jet toward a backstop evacuation channel.

FIG. 1B is another prior art water jet resection device similar to that of FIG. 1A.

FIG. 2A is the working end of a prior art low-pressure water jet resection device that aims the water jet directly at tissue with no backstop.

FIG. 2B is a prior art water jet device similar to that of FIG. 2B.

FIG. 3 Is a View of the various components of the invention comprising a robotic arm, an introducer, a mapping tool, and a resecting-cauterizing device with a block diagram of the controller, liquid jet stream components, vapor jet stream components, and the negative pressure source.

FIG. 4 is a perspective view of the introducer of FIG. 3 separated from the robotic arm.

FIG. 5 is a sectional view of a patient's prostate showing various zones.

FIG. 6 is a sectional view of the prostate of FIG. 5 with the working end of the introducer of FIG. 4 in a deployed position in the prostate.

FIG. 7 is a perspective view of the mapping tool of the invention with sensing needles deployed.

FIG. 8 is an enlarged transparent view of components of the mapping tool of FIG. 7.

FIG. 9A is an enlarged view of the distal tip of a sensing needle of the mapping tool of FIGS. 7 and 8.

FIG. 9B illustrates a variation of a sensing needle.

FIG. 10 is an enlarged view of two instrument drivers of the mapping tool of FIGS. 7 and 8.

FIG. 11 is a sectional view of the prostate of FIG. 6 with the mapping tool introduced through the introducer with the sensing needles deployed,

FIG. 12 is a view of a display of a prostate map algorithmically generated by a controller from data provided by the mapping tool of FIGS. 7 to 1l.

FIG. 13 Illustrates the resecting-cauterizing device of the invention.

FIG. 14 is a perspective view of the working end of the resecting-cauterizing device of FIG. 13, showing first and second water jets.

FIG. 15 Is a transparent view of the proximal housing assembly of the resecting-cauterizing device of FIG. 13.

FIG. 16 is a perspective view of the working end of the resecting-cauterizing device of FIG. 13 in a method of use showing first water jet stream resecting tissue is an exemplary mapped path.

FIG. 17 is a perspective view of the working end of FIG. 16 in a method of use showing first water jet stream resecting tissue is a different mapped path.

FIG. 18 is a variation of the resecting-cauterizing device of FIG. 13, illustrating different instrument drivers.

FIG. 19 is a schematic view of a vapor generator system that generates the water vapor of the second water jet component of FIGS. 13 to 17.

FIG. 20A is a cut-away view of a working end of a water jetting device with a jetting orifice that is adjustable by an instrument driver to propagate the liquid water jet stream in different vectors.

FIG. 20B is a view of the working end of FIG. 20A showing an instrument driver adjusting the propagation vector of the liquid water jet stream.

FIG. 21 is a schematic view of a variation of a working end of a water jet device showing an instrument driver adapted to actuate a variable aperture jetting orifice or nozzle.

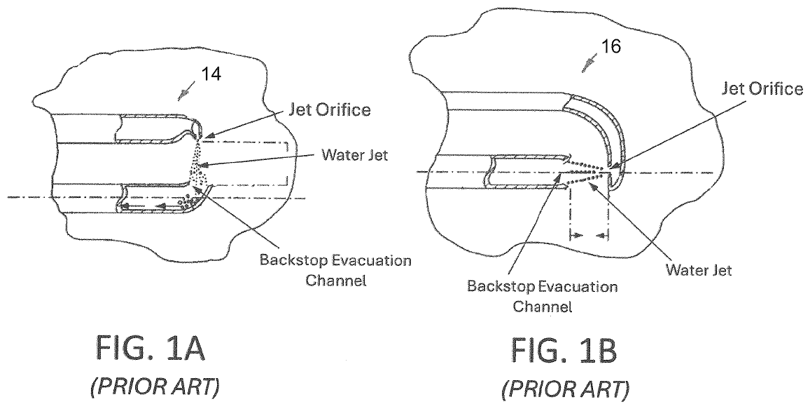

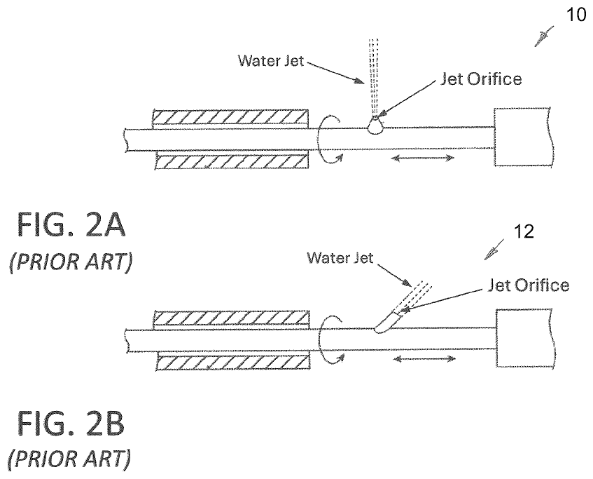

### DETAILED DESCRIPTION OF THE INVENTION

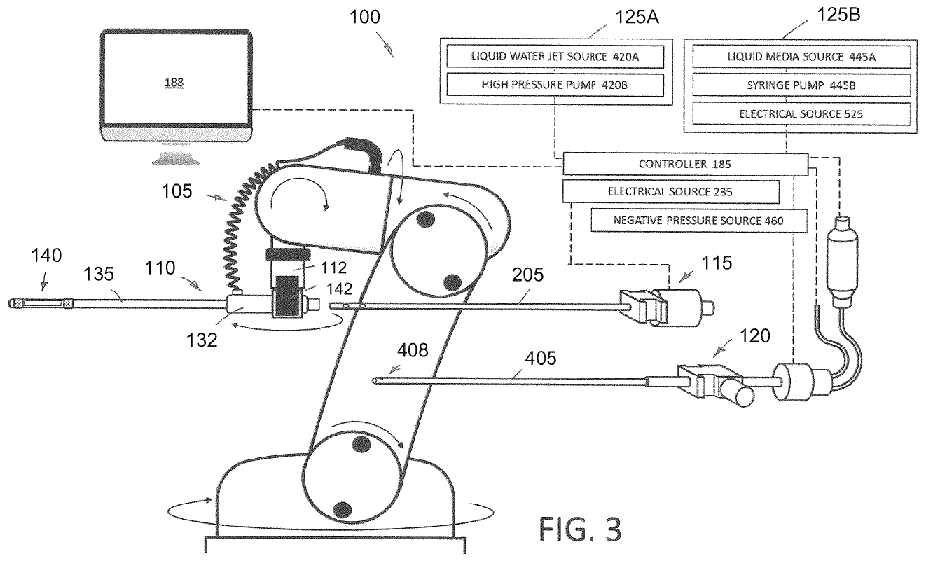

FIG. 3 Illustrates a BPH treatment system 100 corresponding to the present invention and comprises a multi-arm robotic system 105 with a trans-urethral introducer 110 that is detachably coupled to a distal robot arm 112. The introducer 110 is adapted trans-urethral introduction into a prostate and bladder of a patient, after which two different devices are inserted through the introducer 110 to access the patient's prostate. First, a mapping tool 115 is advanced through the introducer 110 to map the prostate and thereafter use the map at the targeted region for resection and extraction. The second device is a resecting-cauterizing device 120 that is advanced through the introducer 110 to volumetrically resect, extract and cauterize prostate tissue. The resecting-cauterizing device 120 incorporates a dual water jet system in a single device that includes a liquid water jet component 125A configured for resecting tissue and a water vapor jet component configured for cauterizing. The introducer 110, mapping tool 115 and resecting-cauterizing device 120 are described in separate sections below.

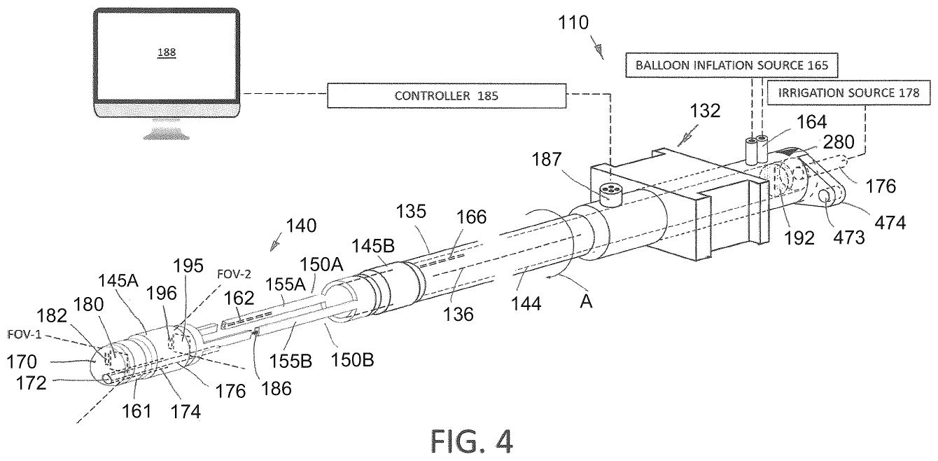

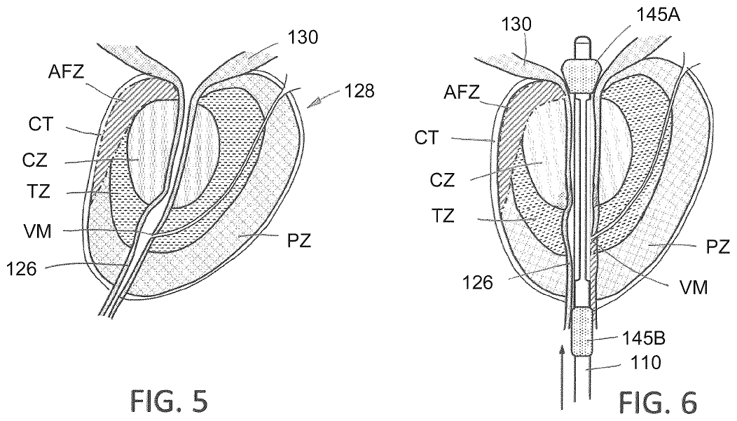

Trans-urethral Introducer in a method of use described below, the introducer 110 of FIGS. 3 and 4 typically is manipulated manually by the clinician to advance the introducer 110 through the patient's urethra 126 to the prostate 128 and bladder 130 (see FIGS. 5 and 6). Thereafter, the clinician couples the hub 132 of the introducer 110 to the distal robot arm 112, which then stabilizes and locks the introducer 110 and robot arm 112 in a selected position. In a variation, the robotic system 105 has moveable arm segments that can be moved freely by the clinician within several degrees of freedom to assume a selected position, and then the robotic system 105 can be actuated to lock the introducer 110 into position. Thereafter a controller, described in detail below, controls the robotic system 105 to manipulate the robot arm segments, instrument drivers, drive elements, and actuators of the mapping tool 115 and the resecting-cauterizing device 120 to resect, extract, and cauterize tissue.

Referring to FIG. 4, the proximal hub 132 of the introducer 110 is coupled to an elongate sleeve 135 extending about longitudinal axis 136 to a working end 140. The hub 132 is detachably coupled to the robotic arm 112 with a suitable latch mechanism 142 (FIG. 3) that may include a drape (not shown) between the hub 132 and latch mechanism 142.

As can be seen in FIG. 4, a working channel 144 extends through the sleeve 135 and is adapted to receive the mapping tool 115 and the resecting-cauterizing device 120 as will be described below. The working end 140 of sleeve 135 carries first and second inflatable occlusion balloons 145A and 145B for anchoring the working end 140 in the patient's urethra 126 as well as for sealing the urethra from unwanted fluid escape during operation of the resecting-cauterizing device 120 as described below. Referring to FIGS. 4 and 6, it can be seen that the working end 140 of sleeve 135 has two sidewall cut-outs 150A and 150B between axial sidewalls 155A and 155B of the sleeve 135. As will be described in detail below, the functional mechanisms of both the mapping tool 115 and the resecting device 120 operate on prostate tissue through the sidewall cut-out or cut-outs 150A, 150B. In typical variations, the working end 140 is configured with one to four axial sidewalls with a corresponding similar number of sidewall cut-outs. The sidewall cut-outs are from 2 cm to 5 cm in length and often 2.5 cm to 4.0 cm in length.

Referring to FIGS. 4 and 6, the working end 140 of sleeve 135 carries a distal occlusion balloon 145A that is positioned near the distal end 161 of sleeve 135. The balloon 145A communicates with an inflation channel 162 in axial sidewall 155A of sleeve 135 that extends proximally to a connector 164 and external balloon inflation source 165 (FIG. 4). Similarly, the proximal occlusion balloon 145B communicates with an independent axial inflation channel 166 in sleeve 135 communicating with the connector 164 and balloon inflation source 165. The balloon inflation source 165 for both balloons 145A, 145B can consist of one or more manually operated syringes or a robotically operated pump mechanism.

The sleeve 135 of the introducer 110 optionally may be encased in a tear-away sheath (not shown) to cover the sidewall cut-outs 155A, 155B. Such a tear-away sheath is a convenient way to provide a very thin wall member to assist in advancing the working end 140 through the urethra 126 of a patient to the prostate 128 and bladder 130 (see FIG. 6).

In FIG. 4, it can be seen that the blunt distal tip 170 of the sleeve 135 has a port 172 therein that comprises the open termination of a channel 174 in the distal tip 170 of the working end 140. It can be understood that for introduction of the sleeve 135 through the patient's urethra 126, the clinician would be aided by endoscopic viewing and irrigation with saline or another liquid. The port 172 and channel 174 communicate with the working channel 144 and are adapted to deliver an irrigation fluid through the working channel 144 and port 172. A removable irrigation tube 176 (dashed line) is provided that extends through the working channel 144 and hub 132 to couple to an irrigation source 178 that can be a gravity system or any suitable positive pressure pump system known in the art. The irrigation tube 176 is removed after the sleeve 135 and working end 140 have been positioned in the patient's prostate 128 to vacate the working channel 144 to receive the shaft of the mapping tool 115. In another variation, a tear-away sheath (not shown) as described above can cover and seal the sidewall cut-outs 150A, 150B, and an irrigation fluid can be delivered directly through the working channel 144 to exit port 172 in the distal tip 170 of sleeve 135.

In a variation as shown in FIG. 4, the endoscopic viewing component carried by the introducer 110 comprises an electronic image sensor 180 with distal field of view FOV-1, and at least one LED 182. In this variation, the image sensor can be an OmniVision model OCHFA10 sensor module or equivalent that integrates the image sensor, a processor, and a lens in a miniature wafer-level module. The image sensor 180 and LED 182 are coupled to controller 185 by a cable or flex circuit 186 extending through axial sidewall 155B to an electrical connector 187 in the proximal portion of sleeve 135, as shown in FIG. 4. The images provided by the image sensor 180 are displayed on display 188.

In FIG. 4, it also can be seen that the introducer sleeve 135 is rotatable (arrow A) in hub 132 to allow radial adjustment of the sidewall cut-outs 150A, 150B as shown in FIG. 6 to allow the clinician to orient the mapping tool 115 and resecting device 120 in a selected direction. FIG. 4 also shows an elastomeric seal 192 disposed in the proximal end of working channel 144 to provide a fluid-tight seal around the shaft of the mapping tool 115 and the resecting device 120 when in use.

In an optional variation, still referring to FIG. 4, a second image sensor 195 with a proximal-facing field of view FOV-2 is provided in working end 140 of the introducer 110. At least one LED 196 also faces the proximal direction. The second image sensor 195 and LED 196 are connected to the cable or flex circuit 186, and electrical connector 187 that is coupled to the first image sensor 180. The second image sensor 195 is thus configured to view in the proximal direction toward resecting device 120 in real-time during use for purposes described in more detail below.

In another variation, a conventional re-usable endoscope (not shown) can be inserted through the working channel 144 for introducing the introducer 110 into the patient's urethra 126 and the image sensors 180 and 195 would not be required. However, large format, high-definition image sensors are inexpensive for single-use devices and offer advantages over re-usable endoscopes that require sterilization and often need repairs.

Mapping tool for mapping prostate capsule. FIGS. 3, 7 and 8 illustrate the robotic mapping tool 115 that includes an instrument driver comprising at least one motor and a drive element comprising at least one sensing needle that are extended and retracted in the prostate to map the capsular dimension of the prostate.

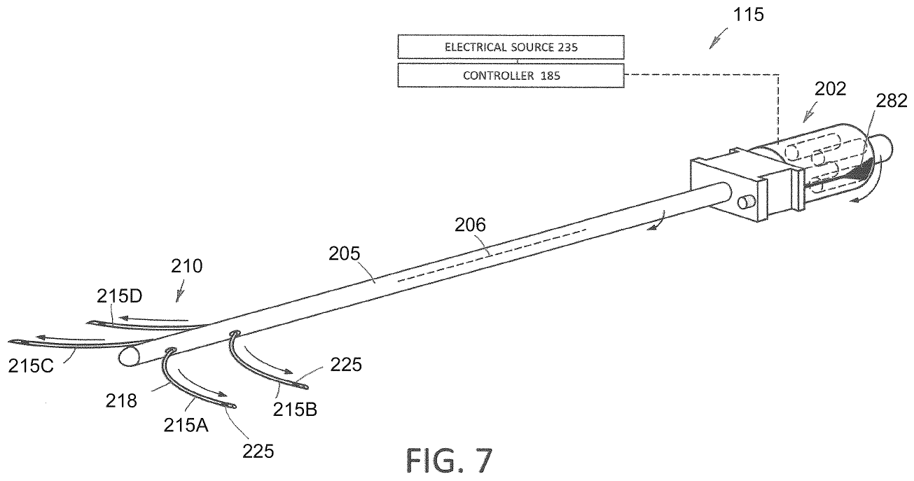

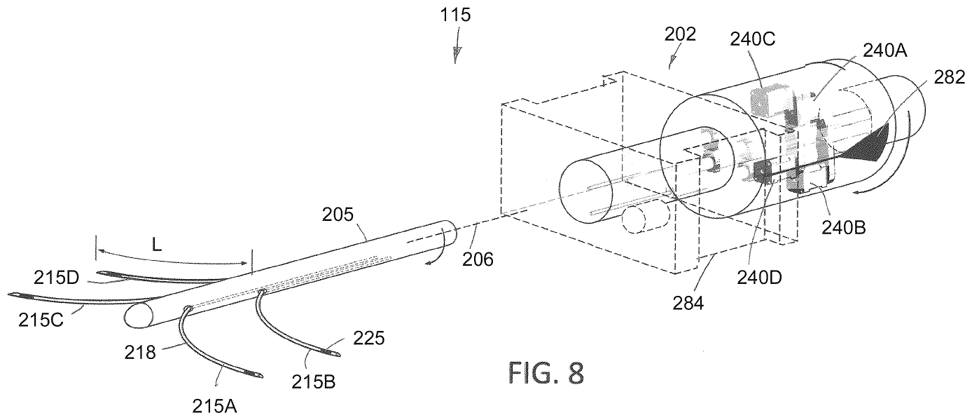

Referring to FIG. 7, the mapping tool 115 is configured for insertion through the working channel 144 of introducer 110 (FIG. 4) after the introducer has been positioned in the patient's prostate and stabilized in place by the robotic system 105 as shown in FIG. 6. Referring to FIG. 7, the mapping tool 115 comprises a proximal housing assembly 202 coupled to an elongated mapping shaft 205 extending about a longitudinal axis 206 to a working end 210 that carries from one to 12 extendable-retractable sensor needles. The variation shown in FIGS. 7 and 8 is configured with four extendable-retractable sensor needles 215A-215D. In this variation, two sensor needles 215A and 215B are extendable from a first side of mapping shaft 205 and the two other sensor needles 215C and 215D are extendable from a second side of mapping shaft 205 that is 180^{o} opposed to sensor needles 215A and 215B.

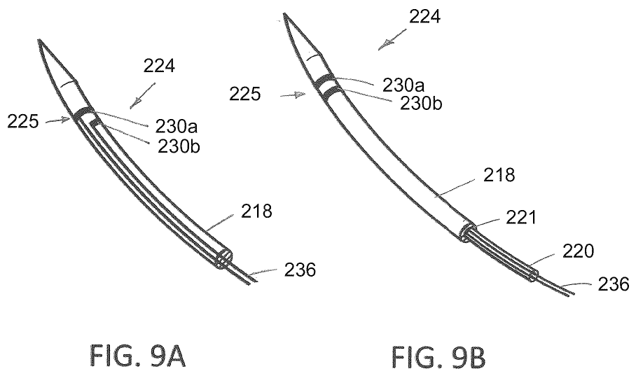

The drive element comprises extendable-retractable sensor needles 215A-215D each comprise a flexible needle shaft 218 that has a curved repose memory shape in a deployed position as shown in FIGS. 7 and 8 that can be deformed into a tensioned straight shape when retracted into the mapping shaft 205. The sensor needle shafts 218 (FIG. 9A) are a solid or tubular polymer material, such as PEEK, with a memory shape as shown in FIGS. 7, 8 and 9A. In another variation, the needle shaft 218 comprises a metal spring core 220 with a polymer coating 221 as shown in FIG. 9B. In FIG. 9B, the metal spring core 220 can comprise a NiTi alloy or similar memory spring material. Each sensor needle 215A-215D has a curved extendable length L (FIG. 8) enabling it to extend outward from the mapping shaft 205 to reach the prostate capsular tissue CT (see FIG. 5). An average normal prostate has a length of 30-40 mm, a width of 20-25 mm and a thickness of 15-20 mm. A prostate in a BPH patient typically will have much larger dimensions and the needle length L along the curved repose shape will be from 20 mm to 30 mm. The needle gauge can range from 16 ga. to 34 ga.

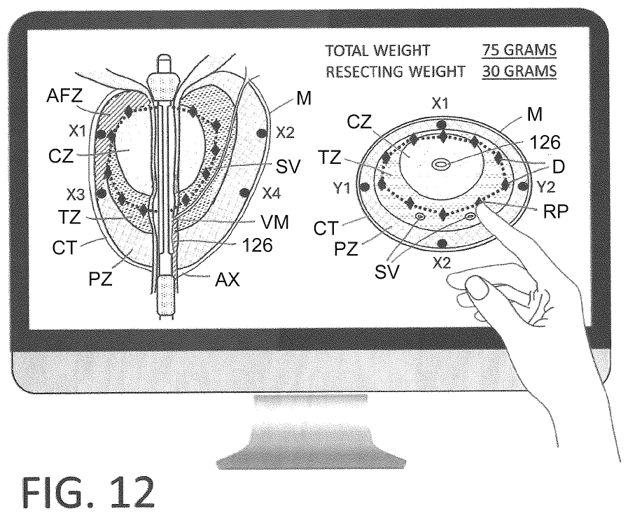

Referring now to FIGS. 7, 8 and 9A-9B, a distal tip 224 of each sensor needle 215A-215D carries a micro-electrode arrangement or sensor 225 that, in a variation, comprises at least two spaced apart electrodes 230a, 230b configured for measuring capacitance and and/or measuring or calculating other dielectric properties of the tissue in contact with the electrode arrangement 225 (FIG. 9A). The controller 185, based on control logic therein, is configured to send an electrical current from electrical source 235 to the electrode arrangement 225 (FIG. 7). The electrical leads 236 extending to the electrode arrangement 225 are formed in trace on the surface of the needle shaft 218 (FIG. 9A) or can be inward of the insulative coating 221 (FIG. 9B). As described in the Summary above, biological tissues have unique capacitance properties due to their composition, as well as other unique dielectric properties that provide distinctive signatures for different types of tissue. In the case of a human prostate 125 as shown in FIG. 5, the tissues in the central zone CZ, peripheral zone PZ, transitional TZ and anterior fibromuscular zone AFZ have unique dielectric properties compared to capsular tissue CT. Based on tissue differentiation, the controller 185 based on control logic and processors therein algorithmically generates a real-time map M of a prostate capsular tissue CT that is displayed on display 188 as described in more detail below (see FIG. 12).

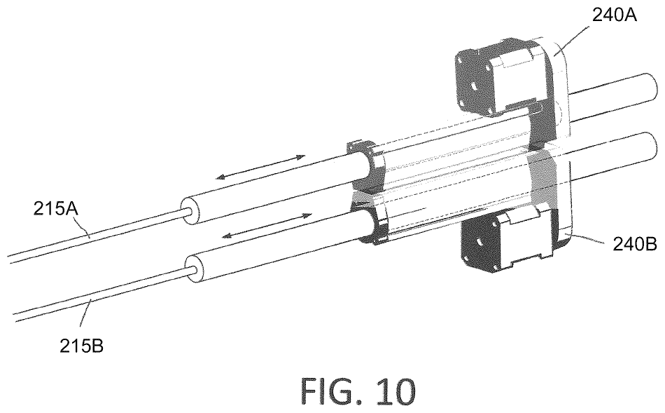

In FIGS. 7, 8 and 10, it can be seen that the sensor needles 215A-215D are extended from a retracted position in the mapping shaft 205 to an extended position outward from shaft 205 by an instrument driver that comprises stepper motors 240A-240D carried in the proximal housing assembly 202. FIG. 10 illustrates an enlarged view of two exemplary stepper motors 240A and 240B that have motors 244A, 244B coupled to the proximal ends of sensor needles 215A and 215B. Such stepper motors are of the type available from Zaber Inc., 605 West Kent Ave. N. Vancouver, British Columbia, Canada. In FIG. 10, each stepper motor 240A, 240B has a worm gear that drives a linear actuator 242A, 242B that each has a spline connection to respective sensor needles 215A, 215B to axially extend and retract the needles.

In this variation, each stepper motor 240A-240D is operated by the controller 185 to advance and retract a respective sensor needle 215A-215D independently. In a variation, each stepper motor 240A-240D is actuated to initially extend a sensor needle outward from the mapping shaft 205 at a very high rate of travel for a distance of 2 mm to 10 mm to penetrate through a wall of the urethra 126 (FIG. 5). Thereafter, each stepper motor 240A-240D advances a sensor needle 215A-215D intermittently or at a slow rate of travel for mapping the prostate. Thereafter, the controller 185 sends electrical current from electrical source 235 to the electrode arrangement 225 on each sensor needle 215A-215D and acquires and processes a capacitance measurement and/or other dielectric measurement. The processor algorithms in controller 185 then compare the dielectric measurements to known dielectric values in a library and determines if the electrode arrangement 225 in each sensor needle 215A-215D is in contact with capsular tissue CT or inner glandular tissue in other zones of the prostate. Alternatively, each sensor needle 215A-215D is extended outward at a selected slow rate of travel, and dielectric measurements can be processed continuously as the needles 215A-215D are advanced toward the prostate capsular tissue CT. In either method of needle advancement, when the dielectric signals indicate that the electrode arrangement 225 of any sensor needle is in contact with capsular tissue CT, the location of the needle tip 224 relative to the mapping shaft 205 and introducer 110 is mapped onto the display 188 as shown in FIG. 12. Visual and audio alerts can inform the clinician when any needle contacts capsular tissue CT.

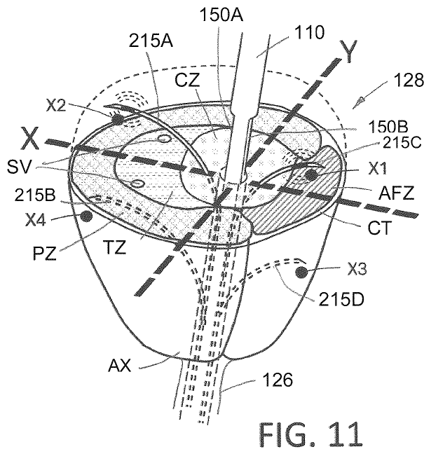

FIG. 11 is a sectional perspective view of prostate showing the advancement of sensor needles 215A to 215D outward in sagittal plane X in the patient's prostate 128. It can be seen that the sensor needles 215A to 215D extend outward from the sidewall openings 150A-150B of the introducer 110 where the sensor needles 215A-215D reach capsular tissue CT at respective points X1 to X4. Thereafter, the points X1 to X4 are mapped to create a visual map M of the prostate capsular tissue CT on display 188, as shown in FIG. 12.

Following the mapping of X1 to X4, as shown in FIGS. 11 and 12, the sensor needles 215A-215D are retracted into the mapping shaft 205, and thereafter, the introducer 110 and mapping tool 115 are rotated 90° as can be understood from FIG. 11 to then map the prostate 128 in coronal plane Y. The needle advancement steps and sensing steps described above are repeated to determine the locations of the capsular wall tissue CT in plane Y and then mapped to the display 188, as shown in FIG. 12. It can be understood that each stepper motor 240A-240D signals the controller 185 of each dimensional extension of each needle shaft 215A-215D to contact capsular tissue CT is known, together with the curvature of each needle in soft glandular tissue in the central zone CZ, transitional zone TZ and peripheral zone PZ (see FIGS. 5 and 6) and allows for very accurate mapping of the dimensions and volume of the patient's prostate 128. In the above variation of mapping tool 115 of FIGS. 7 and 8, four sensor needles 215A-215D are deployed in sagittal and coronal planes, but it should be appreciated that one to four such sensor needles can be extended outward in opposing sides of mapping shaft 205 to acquire locations of the capsular tissue CT. Further, the sensor needles can be deployed in additional planes intermediate the sagittal and coronal planes to provide additional mapping points. However, it is believed that accurate mapping of a prostate 128 in combination with a library of prostate dimensional data will provide highly accurate images of a prostate 128 to allow for programming a resecting device 120 as described below for any volumetric reduction selected by the clinician.

Referring to FIGS. 6 and 7, it can be understood that the mapping tool 115 and the introducer 110 must be radially oriented so that the sensor needles 215A-215D extend outward from the sidewall cut-outs 150A-150B. For this reason, it can be seen in FIGS. 4, 7, and 8 that the proximal housing 132 of the introducer 110 has a marking 280 and the proximal housing assembly 202 of the mapping tool 115 has a marking 282 that can be aligned to insure the proper orientation. In the variation of FIGS. 4-7, the rotation of the mapping tool 115 in introducer 110 between planes X and Y, as understood from FIG. 11, is done manually, but the housing assembly of the mapping tool 115 also has engagement features 284 that allow for robotic latching mechanism to grip the mapping tool 115 to allow a robotic mechanism to automate rotation of the mapping tool 115 together with the introducer 110.

Referring to FIG. 12, it Can be understood that the processors and controller 185 generate a visual map M of the capsular tissue CT of a patient's prostate 128 that can be viewed by the clinician in any desired sectional sagittal, transverse, and coronal view for evaluation. In a variation, the display 188 is a touch screen display wherein the clinician then touches the screen at a plurality of points D indicated by diamond-shaped elements to define the contours or shape of a selected resection profile RP. The resection profile RP in FIG. 12 is then selected by the clinician to be inward from the displayed positions X1-X4 and Y1-Y4 of the capsular tissue CT to provide a margin of safety, which may be inward from 2 mm to 5 mm or more. Further, the processed image of the prostate in FIG. 12 includes the location of the verumontanum VM in the patient's prostate 125. The location of the verumontanum VM is not acquired by the mapping tool 115 but rather is noted by the clinician during the initial step of advancing the introducer 110 through the urethra 126. During this step of the procedure, the clinician makes note of the location of the verumontanum VM from the apex AX of the prostate 128 and then later uses a grid or caliper on the touch screen 188 to mark the location of the verumontanum VM. The controller 185 then can access a data library of prostate profiles to estimate the location of the seminal vesicles SV that are mapped as shown in FIG. 12.

In a variation shown in FIG. 12, the controller 185 and processors therein can calculate and display the volume of tissue that would be resected within the resection profile RP selected by the clinician. In FIG. 12, the total prostate volume in grams is displayed based on the acquired points X1-X4 and Y1-Y4 of capsular tissue CT, and the resection volume is then calculated from the selected resection profile RP selected by the clinician. In a typical method of selecting a resection profile RP, the clinician selects a profile the does not resect the verumontanum VM and further may protect the seminal vesicles SV. Thus, a clinician may typically select a resection profile RP, as shown in FIG. 12 that resects tissue only in the direction of the bladder 130 away from the verumontanum VM and is deeper in the posterior direction and shallower in the anterior direction, In another variation, the clinician may select a resection profile RP that resects tissue towards the apex AX side of the verumontanum VM while preserving the verumontanum VM.

The above variation of FIGS. 7-10 describe stepper motors that are configured to extend and retract the four sensor needles 215A-215D. It should be appreciated that other mechanisms can be used to initially penetrate any number of sensor needles through the wall of the urethra 126, such as releasable spring mechanisms or magnetic repelling mechanisms. Such needle penetration mechanisms are known in the art and can be manually or robotically operated and fall within the scope of the invention.

In another aspect of the invention, a secondary redundant mechanism is used to map the capsular tissue CT of the prostate in combination with the dielectric sensors described above. It can be understood from FIGS. 7 and 8 that each of the stepper motors 240A-240D is actuated by the controller 185 to slowly or intermittently advance each sensor needle 215A-215D through prostate tissue at a selected rate of advancement. The glandular tissue in inner zones of the prostate 128 is similar in density, and the capsular tissue CT is far more dense and is resistant to penetration by a sensor needle. Therefore, the controller 185 is provided with algorithms that can monitor voltage at each stepper motor required to advance each sensor needle at the selected rate. When the voltage increases to enable a stepper motor to drive the needles through denser tissue, it indicates that capsular tissue is being approached. Therefore, the controller 185 can further correlate the voltage being used with tissue density measurements to differentiate inner glandular prostate tissue from capsular tissue CT. Such a tissue density determination can be combined the dielectric measurements to confirm the depth at which capsular tissue CT has been contacted be each the sensor needle tip 224.

In a variation, the assembly carrying stepper motors 240A-240D comprises a multiple-use assembly and is coupled to a single-use assembly comprising the shaft 205 and sensors needles 215A-215D. Further, a drape (not shown) can be positioned between the single-and multi-use assemblies with coupling mechanisms known in the art for coupling instrument drivers with drive elements.

In general, the robotic medical system for mapping tissue comprises an instrument driver and a drive element comprising at least one needle operatively coupled to the instrument driver, a controller 185 having control logic configured to operate the instrument driver and move the drive element through a patient, and a dielectric sensor integrated into the at least one needle and configured to acquire and send data to the controller, wherein the acquired and processed data is used by the controller, based on the control logic, to control the instrument driver and to actuate the dielectric sensor. In a variation, the controller 185 is configured to extend the at last one needle along a path and actuate the sensor continuously or intermittently until a dielectric value of tissue in contact with then senor indicates capsular tissue and then the instrument driver retracts the at least one needle. In a variation, the controller rotated the mapping tool 115 and optionally the introducer to map the tissue is a different plane in the prostate.

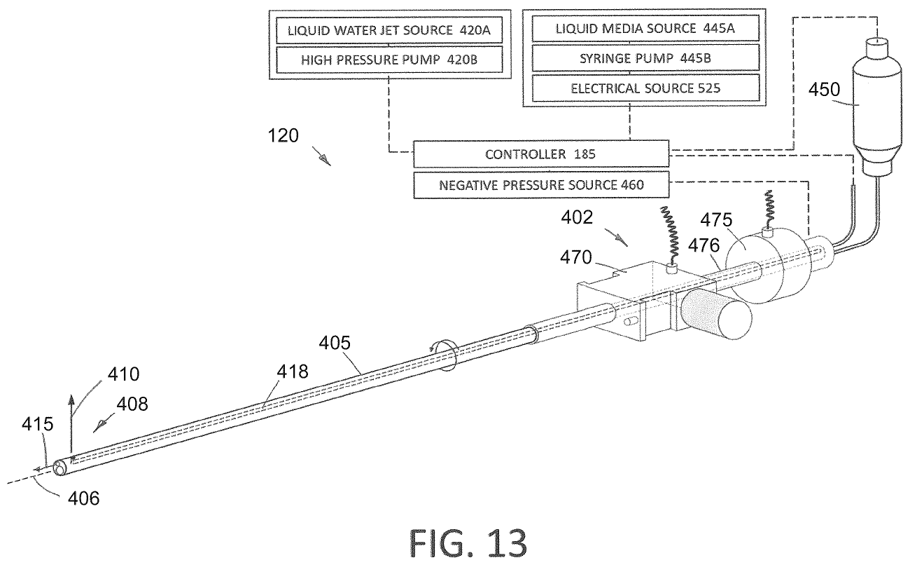

Resecting-cauterizing device. FIGS. 13-15 illustrate the resecting-cauterizing device 120 comprising a proximal hub assembly 402 coupled to an elongate jetting shaft 405 extending about axis 406 that is adapted for advancing through the working channel 144 in the introducer 110 when positioned in a prostate as shown in FIG. 6. The working end 408 of the shaft 405 carries a dual water jet system that comprises (i) a first water jet component 125A comprising a liquid-state water or saline that is jetted outward in a liquid water jet stream 410 that carries kinetic energy capable of resecting prostate tissue, and (ii) a second water jet component 125B comprising a vapor-state water that is jetted in a water vapor stream 415 that undergoes a vapor-to-liquid phase change in the prostate to thereby apply the heat of vaporization to cauterize tissue. The water vapor stream 415 will cauterize an exposed surface of tissue after (or before) the volumetric resection and extraction of such tissue corresponding to the resection profile RP as described above.

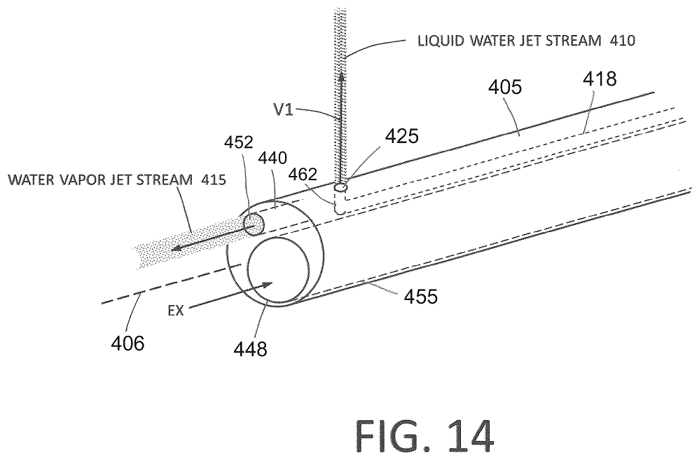

In FIGS. 13 and 14, it can be seen that jetting shaft 405 is configured with a first axial jetting channel 418 that communicates with remote liquid water source 420A and high-pressure pump 420B to deliver a high-pressure liquid water jet stream 410 from the water jet orifice 425 in the working end 408. The jetting shaft 405 is also configured with a second axial jetting channel 440 that communicates with remote liquid media source 445A and pump 445B that delivers liquid water to a vapor generator 450 (FIG. 13) that converts the liquid water to water vapor and pumps the water vapor though the second jetting channel 440 to a vapor jet orifice 452 in the jetting shaft 405 as further described below.

Referring again to FIGS. 13 and 14, the jetting shaft 405 also is configured with a third axial channel comprising a distal port 448 and extraction channel 455 that communicate with remote negative pressure source 460 for aspirating fluid and resected tissue debris from a treatment site in the direction of arrow EX (FIG. 14). The extraction channel 455 and its connection to negative pressure source 460 is also further described below.

First water jet component 125A for resecting tissue. Referring to FIGS. 13 and 14, the axial jetting channel 418 in jetting shaft 405 transitions to a short radial channel 462, leading to the water jet orifice 425 in an outer surface of the jetting shaft 405. The high-pressure pump 420B thus provides a high-pressure liquid water jet stream 410 that propagates outward of water jet orifice 425 about vector V1 that is approximately 90^{o} from the axis 406 of the jetting shaft 405.

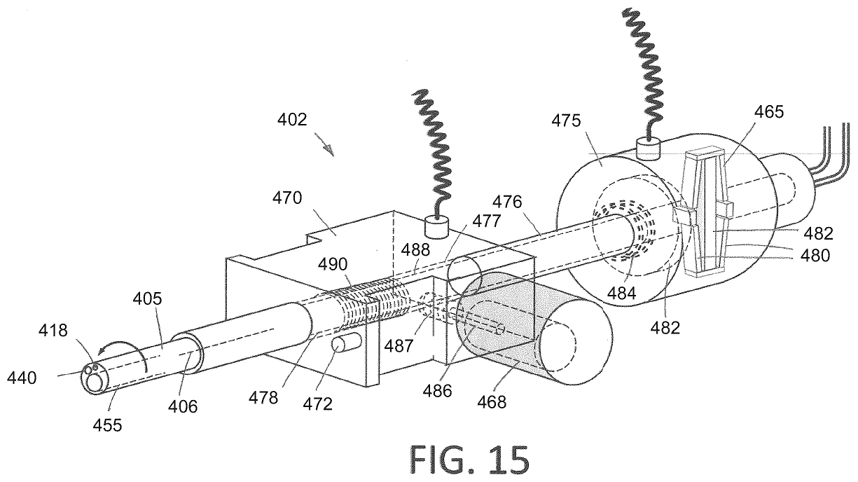

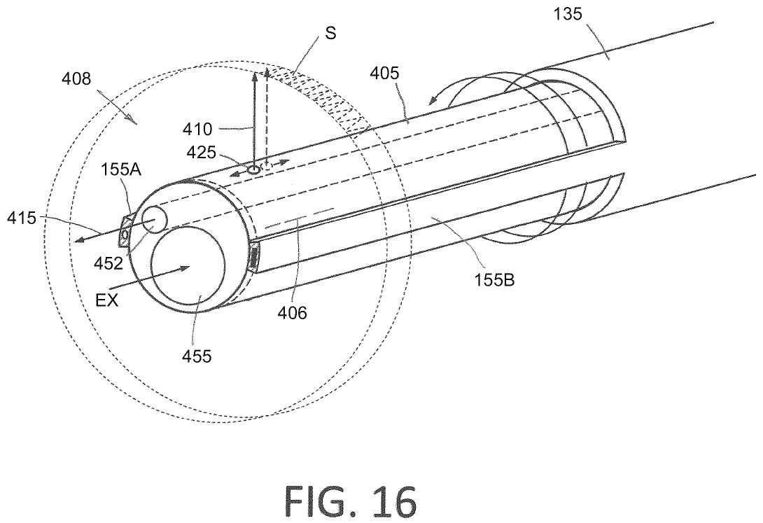

FIG. 15 illustrates the proximal hub assembly 402 of the device 120 that includes first and second actuation mechanisms for moving the jetting shaft 405. The first actuation mechanism comprises a stacked piezoelectric actuator 465 configured to jitter or oscillate the jetting shaft 405 to thereby jitter the liquid water jet stream 410 (FIG. 14) at a high speed. The second actuation mechanism comprises a motor drive 468 configured to helically advance and retract the jetting shaft 405 within the working channel 144 of the introducer 110 (see FIGS. 6 and 16).

In FIGS. 13 and 15, it can be seen that the drive housing 470 of the hub assembly 402 is configured with pin 472 to engage bore 473 in flange 474 on the introducer sleeve 110 (FIG. 4) or a latching mechanism on an arm of the robotic system 105 alternatively is adapted to grip and stabilize the drive housing 470 in the introducer sleeve 110. A second housing or jitter housing 475 is fixed to an active sleeve 476 that is helically movable through an axial channel 477 in the drive housing 470. The jetting shaft 405 is moveable in a bore 478 in the drive housing 470, contemporaneous with the helical movement of the active sleeve 476. The diameter of the jetting shaft 405 and active sleeve 476 are selected to fit with the working channel 144 of the introducer sleeve 110 (FIG. 4), wherein the working channel 144 can have a larger diameter in the proximal housing assembly 132 and a reduced diameter in the elongated sleeve 135.

Referring to FIGS. 13 and 15, the jitter housing 475 of the hub 402 carries the piezoelectric actuator 465 that is adapted to axially jitter or oscillate the jetting shaft 405 during use. In a variation, the piezoelectric actuator 465 (not-to-scale) interfaces with a flange 482 coupled to the jetting shaft 405. In a variation, the stroke of the piezoelectric actuator 465 is enhanced by a parallelogram-type flexure amplifier elements 480 that are configured to amplify the displacement of the piezoelectric element stack 465 by a factor of 10× to 50×. Such an actuator 465 can provide a stroke of up to 4.0 mm, and often the actuator 465 will have a stroke of between 0.25 mm 2.5 mm. The stacked piezoelectric actuator 465 operates at frequencies between 1 Hz to 20 Hz, which provides jitter or oscillations in the liquid water jet stream 410. A spring 484 is shown in FIG. 15 between the flange 482 and the housing 475 that urges the jetting shaft 405 in the proximal direction as the piezoelectric actuator 465 actuates the jetting shaft 405 in the distal direction. It should be appreciated that linear stacked piezoelectric actuators may be used without a flexure amplifier or other type of amplifier and may be suitable for providing the jitter. Piezoelectric stack actuators with or without flexure amplifiers are available, for example, from Thorlabs, Inc., 43 Sparta Ave, Newton, New Jersey 07860.

FIG. 15 further shows motor drive 468 carried in the drive housing 470 of the hub 402. The motor drive 468 has a drive shaft 486 with a gear 487 that engages cooperating gear surface 488 on active sleeve 476. The helically actuated active sleeve 476 is configured to function as helical lead screw in threads 490 in the housing 470 to thus contemporaneously helically advance or retract the jetting shaft 405. Thus, the motor drive 468 will move the liquid jet orifice 425 in a sweeping helical path around axis 406 of the shaft over 360°. In operation, FIG. 16 shows the actuation of both the piezoelectric actuator 465 and the motor drive 468 to cause the working end 408 and liquid water jet stream 410 to sweep around that axis 406 of the working end 408 to cut and extract tissue.

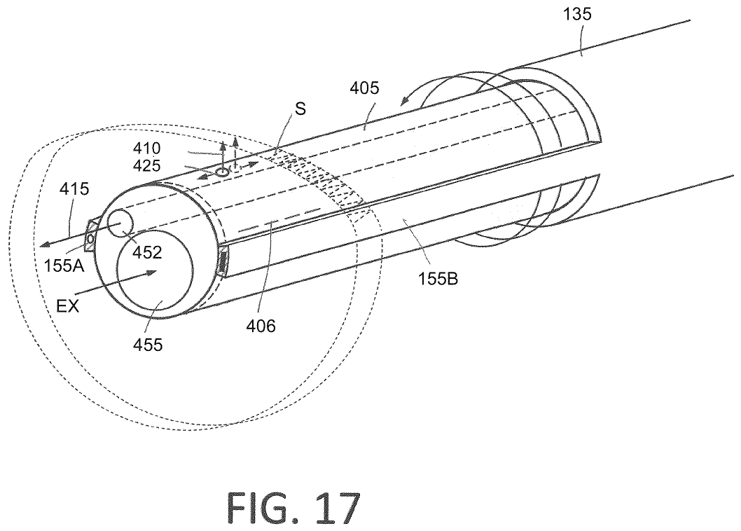

In FIGS. 16 and 17, it Can Be understood that the controller 185 is configured to modulate four operating parameters of the system to control the tissue cutting or disintegration effects, which thus can achieve the function of a “backstop” to control cutting depth of the liquid jet stream 410. The controller 185 is configured to control operating parameters including (i) the pressure of the liquid water jet stream 410, (ii) the jitter rate provided by the piezoelectric actuator 465, (iii) the stroke provided by the piezoelectric actuator 465, and (ii) the rate of movement of the jetting shaft 405 by the motor drive 468. Thus, the controller 185 is capable of controlling toward a shallower cutting depth by (i) modulating toward a lower pressure of the liquid jet stream, (ii) modulating toward a higher jitter rate, (iii) modulating toward a larger piezoelectric stroke, and (iv) modulating toward a higher rate of movement of the jetting shaft 405 to reduce the cutting depth. In order to modulate toward and increase cutting depth of the liquid water jet stream 410, the controller 185 would modulates the operating parameters in the opposite directions from this described above. It should be appreciated that the controller 185 also controls negative pressure from the negative pressure source 460 that, is selected to cooperate with the modulation of the other operating parameters and plays a role in controlling cutting depth.

In the example of FIG. 16, the controller 185 maintains a constant pressure in delivering the liquid water jet stream 410, maintains a constant jitter rate, maintains a constant jitter stroke, and maintains a constant rate of the movement of the jetting shaft 405 by the drive motor 468, thus resulting in a uniform cutting depth around axis 406 of the working end 408.

As described above, the jitter stroke S in FIGS. 16 and 17 is often from 0.25 mm to 2.5 mm and the pitch of the leads screw threads 490 that advances and retracts the jetting shaft 405 assembly can be less than the jitter stroke to thus causes the swept cutting paths to overlap after 360° helical advancement of the jetting shaft. FIGS. 16 and 17 show the jetting shaft 405 rotating within the axial sidewalls 155A, 155B of the introducer 110 (see FIG. 4). In a variation, the controller 185 terminates or modulates the pressure of the liquid water jet stream 410 as it passes the axial sidewalls 155A and 155B. In another variation, the pressure and speed of helical movement of the jetting shaft 405 would prevent the water jet stream from damaging the sidewalls 155A and 155B.

It can be understood that the motor drive 468 comprises a stepper motor that sends signals to the controller 185 related to the helical movement of the jetting shaft 405 from a “home base” starting position recorded by the controller 185. Thus, since the pitch of the lead screw thread 490 is known, the exact location of the water jet orifice 425, both radially and axially, can be determined at any point in time by the controller 185.

FIG. 17 Illustrates the controller 185 modulating the liquid water jet stream pressure, the jitter rate, the jitter stroke S, and the speed of the helical movement of the jetting shaft 405 to resect shallow resection at certain radial zones and deeper resection in other selected zones. It can be understood that any resection profile RP, as shown in FIG. 12 can be achieved with the system.

In a variation, the portion of the proximal housing 402 carrying the piezoelectric actuator 465 and the motor drive 468 comprises a multiple-use assembly and is coupled to a single-use assembly comprising the jetting shaft 205. Further, a drape (not shown) can be positioned between the single and multi use assemblies with coupling mechanisms known in the art for coupling instrument drivers with drive elements.

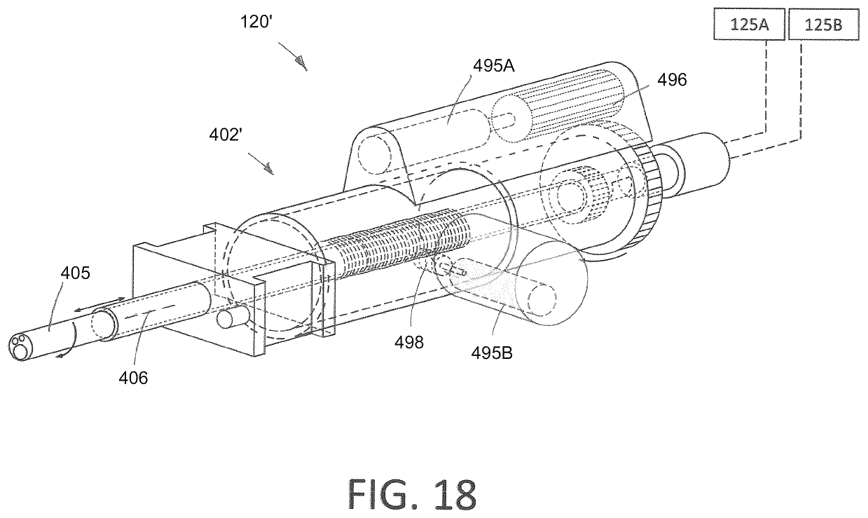

In another variation shown in FIG. 18, the resecting device 120′ has a proximal housing 402′ that carries a first motor drive 495A and a second motor drive 495B. The jetting shaft 405 is the same as described previously. In this variation, the first motor drive 495A has a gear mechanism 496 that independently rotates the jetting shaft 405. The second motor drive 495B has a gear mechanism 498 configured to independently move the jetting shaft 405 axially in distal and proximal directions. This variation configured with first and second motor drives 495A, 495B allows the controller 185 to control another variable, which is not possible with helical advancement and retraction of the jetting shaft as in the variation FIGS. 13-17. For example, the controller 185 can provide for high-speed rotational sweeps around the axis 406 and slow advancement and retraction along the axis 406, or vice versa.

In general, the robotic medical system comprises an instrument driver and a drive element comprising a treatment tool with a working end configured to propagate a high-pressure liquid water jet stream in a patient wherein the instrument driver comprises a piezoelectric actuator operatively coupled to the working end and a controller that, based on control logic, is configured to control both movement and actuation of the working end and liquid water jet stream in within the patient to resect tissue. The piezoelectric actuator comprises a stacked piezoelectric actuator, and, in a variation comprises a flexure-amplified stacked piezoelectric actuator. The piezoelectric actuator has a stroke of at least 0.25 mm to 4.0 mm and often has a stroke ranging from 0.25 mm to 2.5 mm.

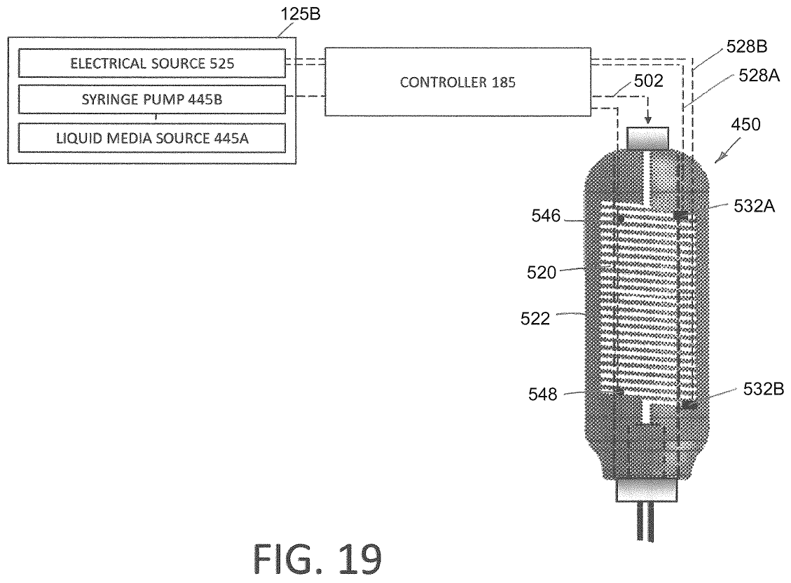

Second water jet component 125B for cauterizing tissue. Referring to FIGS. 13 and 19, the resecting-cauterizing device 120 includes the second water jet component 125B that includes liquid media source 445A, syringe pump 445B, and vapor generator 450 to provide the water vapor jet stream 415 that propagates outward of vapor jet orifice 452 in the jetting shaft 405 in the distal direction as shown in FIGS. 14, 16 and 17.

FIG. 19 is an enlarged schematic view of the components of the vapor generator 450 illustrating the controller 185 configured to control the syringe pump 445B to deliver liquid media from the liquid media source 445A through tubing 502 to the vapor generator 450. The vapor generator 450 comprises a heating element 520 in the housing 522. The housing 522 is detachably mounted on an arm of the robotic system 105 or alternatively can be detachably coupled to the proximal jitter housing 475 of the device 120. In a variation, the heating element 520 comprises a metal that can be resistively heated and can be coiled, straight, or a combination thereof to provide a suitable shape for fitting in housing 522. In FIG. 19, the heating element 520 is formed into a helical configuration to provide a compact form factor for fitting in housing 522. In a variation, a direct current (DC) electrical source 525 generates an electrical current that is coupled to the heating element 520. In other variations, the DC current can be supplied from a battery, for example, a 12 Volt or 24 Volt DC power supply. FIG. 19 illustrates electrical leads 528A and 528B are connected to the heating element 520 at connections 532A and 532B, respectively, on opposing ends of the helical-formed heating element 520.

It can be understood that the design parameters of the syringe pump 445B and liquid flow rates, the heating element 520, and the electrical source 525 are inter-related, and in general, a typical variation is designed to provide a selected calories/second rate of applying energy to tissue that is optimal for cauterization of prostate tissue. In general, the inter-related design parameters include (i) a selected ml/min of liquid media flow within the helical heating element 520, which is further dependent on flow channel diameter, flow channel length, and flow pressure; (ii) the power delivered by the electrical source 525 which further relates to helical tubing design and materials, and ultimately results in a selected vapor quality, i.e., the percent of the flow exiting vapor jet orifice 452 that is phase changed to pure vapor as opposed to non-phase changed liquid droplets. In a variation, system provides a flow of vapor that is greater than 80% water vapor or greater that 90% water vapor and further provides an ultimate conversion efficiency of electrical energy to vapor energy of at least 60%.

In a variation, the controller 185 operates the electrical source 525 to deliver at least 100 W, together with delivering sterile water as a liquid media with pump 445B at a flow rate of between 1 ml/min and 5 ml/min into the helical heating element 520 having a flow channel with a diameter of 0.05″ and a length 50 cm with the helical tubing portion having a diameter of 10 mm. In another variation, the controller 185 can be configured to monitor voltage across the heating element 520, and the current through the heating element 520 can be determined to provide an accurate, real-time measurement of power being dissipated into the fluid flow and the heating element 520. The delivery of the water vapor stream 415 can deliver up to 540 cal/gm that is released by the vapor-to-liquid phase change that in turn can instantly cauterize the exposed surface of resected tissue in the prostate 128 (FIG. 6).

In a variation, the interior flow channel of the helical heating element 520 has a diameter between 0.02″ and 0.10″ and a flow channel length of between 20 cm and 200 cm. The outside diameter of the helical heating element 520 as an assembly is from 5 mm to 20 mm. In a variation, the helical heating element 520 is formed of a stainless steel, Inconel, or any other suitable resistively heatable metal. In a variation shown in FIG. 19, the helical heating element 520 carries at least one temperature sensor coupled to controller 185 and is shown in FIG. 19 with two temperature sensors 546 and 548 at proximal and distal ends of the helical heating element 520. In another variation, a third temperature sensor (not shown) is carried in a medial portion of the helical heating element 520. The plurality of temperature sensors is adapted to send temperature signals to the controller 185, and thereafter, wherein the controller 185 in response to the temperature signals modulates operation of the pump 445B and its flow rate and/or the energy delivered from electrical source 525 to insure the generation of high-quality water vapor stream 415 is provided by the heating element 520 which then results in the desired cal/sec delivered from the vapor delivery orifice 452 (FIG. 14).

In a variation shown in FIG. 12, the display 188 comprised a touchscreen display operably connected to the controller 185 and the second water jet component 125B and allows the clinician to select operating parameters of the vapor generating system, display alerts, and provide other operating information.

In FIG. 14, the water vapor stream 415 exits the vapor jet orifice 452 in the distal tip of the jetting shaft 405. It should be appreciated that the vapor jet stream 415 can cauterize the exposed surface of targeted tissue no matter the location of the vapor jet orifice 452 in the working end 408. For example, the vapor flow channel 418 and vapor jet orifice 452 can be positioned in one or more surfaces of the jetting shaft 405. In another variation, a vapor flow channel and a vapor jet orifice (not shown) can be carried in the introducer 110 (see FIG. 4).

In a method of using the integrated resecting-cauterizing device 120, the delivery of the water vapor stream 415 to cauterize tissue can be used in one of several ways to cauterize tissue in a BPH resection procedure. For example, component 125B can provide a vapor jet stream 415 to thermally seal and coagulate tissue before tissue resection with liquid jet stream 410 is commenced. In such a method variation, the liquid water jet stream then only resects tissue that has already been thermally coagulated and sealed. In another variation, the automated liquid water jet stream resects tissue and thereafter, a vapor jet stream 415 is delivered for a selected time interval to accomplish the cauterization step. In yet another variation, the liquid jet stream resection and delivery of vapor streams 410, 415 is sequential, with a liquid jet stream resection interval of 5 seconds to 30 seconds followed by vapor jet delivery for 5 to 30 seconds, with the intervals repeated multiple times until the targeted volumetric reduction is achieved, In yet another variation, the liquid water jet stream 410 is delivered contemporaneously with the water vapor jet stream 415 that cauterizes the tissue surface during the resection.

In another aspect of the invention, the automated robotic resection is assisted with artificial intelligence (AI) and/or machine learning. The controller 185 is provided with algorithms adapted to monitor video imaging from the device's image sensor 195 in an introducer 110 of the type shown in FIG. 3 and the image processor in real-time identifies selected site characteristics or resecting artifacts within view of the image sensor 195. In response to the observation of a selected characteristic or resecting artifact, the controller 185 automatically modulates, commences or terminates an operating parameter of the system, wherein examples of operating parameters modulation of the liquid water jet stream 410, the water vapor jet stream 415, the pressure of the liquid water jet stream 410, the jitter rate, the jitter stroke, adjustment of cal/sec delivered by the water vapor jet stream 415, movement of the jetting shaft 405, modulation of the negative pressure source 460, and adjustment of the position of arms of the robotic system 105.

The site characteristics or artifacts that the AI or machine learning algorithms monitor are, at a minimum: image observable colors that indicate bleeding, observable bubbles in images that indicate cavitation, observable collapse of side walls of the resection cavity, observable tissue debris that indicates sub-optimal cutting, color of tissue indicating cauterization or coagulation, tissue features indicating prostate tissue types, and identification of verumontanum, ducts and other distinguishing features of a prostate.

In general, a robotic medical method comprises introducing a working end of a resecting system into a prostate, wherein the working end carries an image sensor, a jetting orifice for jetting a first water jet stream that carries kinetic energy capable of resecting tissue and a second water vapor jet stream that undergoes a vapor-to-liquid phase change capable of cauterizing tissue, propagating the first water jet stream thereby resecting tissue operating a controller to monitor video images from the image sensor and comparing artifacts in the video images with a library of resecting artifacts, and operating a controller, based on control logic, to adjust an operating parameter of the resecting system responsive to observing a resecting artifact.

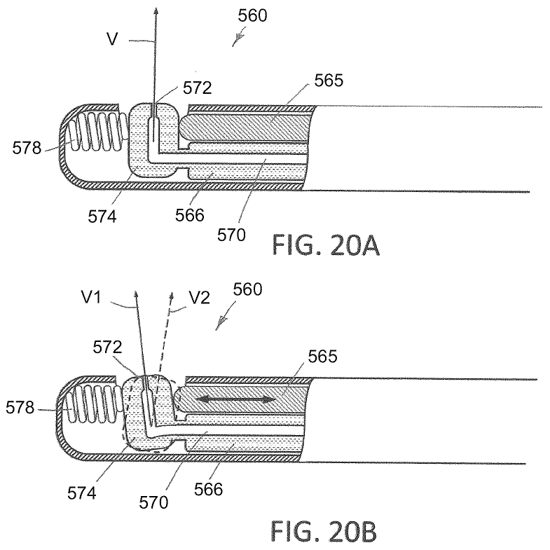

FIGS. 20A and 20B illustrate another variation of a working end 560 of a water jet device with an instrument driver that actuates an actuator member 565 to adjust the propagation vector V of a liquid water jet stream during use. In FIG. 20A, it can be seen that jetting shaft 566 comprises a flexible material such as PEEK that carries an inflow channel 570 extending to the jetting orifice or nozzle 572 in a jet housing 574. The jet housing 574 coupled to the jetting shaft by a flexible element 575 that allows the jet housing 574 to wobble. As can be seen in FIG. 20B, the actuator member 565 is adapted to move axially in the range of 0.25 mm to 2.5 mm to actuate or wobble the jet housing 574 to thus wobble liquid jet stream between propagation vectors V1 and V2. A spring 578 is provided to urge the jet housing 574 back to a repose position. In this variation, the instrument driver that drives the actuator member 565 is a piezoelectric actuator as described above or a motor drive that uses any suitable reciprocating mechanism as known in that art.

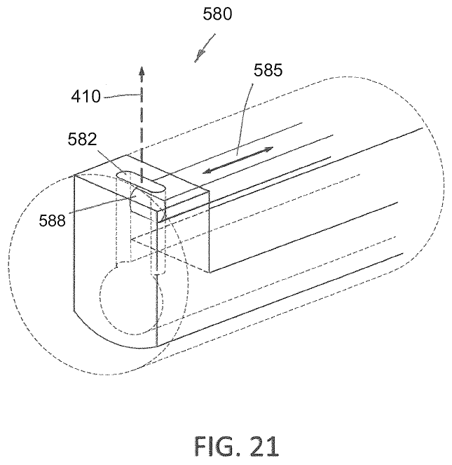

FIG. 21 is a schematic view of another variation of working end 580 that is adapted to vary the dimension of the jetting aperture or nozzle 582. In this variation, an elongate shaft 585 again is operated by a piezoelectric actuator or a motor drive to reciprocate the shaft 585. It can understand that the distal tip 588 of the shaft 585 can then impinge upon jetting aperture 582 to change the shape and cross-sectional dimension of the jetting aperture 582 to vary operating characteristics of the liquid jet stream propagating from the jetting aperture.

The above methods have been described with reference to cauterizing prostate tissue after tissue removal to treat BPH, but it should be appreciated that other prostate treatments may require tissue resection followed by cauterization, such as prostate cancer treatment. While the systems have been described in BPH, treatments, the tools can be used in other mapping, resection and cauterization procedures.

The methods described above refer to the use of condensable water vapor, but other vaporizable liquids may be used, such as vaporized saline or vaporized alcohol.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications and patents cited herein are hereby incorporated by reference as if set forth in its entirety herein.

1. A surgical method for resecting tissue, comprising:
introducing a treatment device into a treatment site in a patient, wherein a working end of the treatment device is configured to deliver a first water jet stream and a second water jet stream into the treatment site;
propagating the first water jet stream comprising a high-pressure liquid water jet stream that carries kinetic energy thereby resecting tissue in the treatment site; and
propagating the second water jet stream comprising a water vapor jet stream that undergoes a vapor-to-liquid phase change in the treatment site thereby applying a heat of vaporization to tissue to cauterize the treatment site.

2. The surgical method of claim 1, wherein the treatment device is coupled to at least one instrument driver, further comprising operating a controller, based on control logic, to operate the at least one instrument driver to control both movement and actuation of the working end within the treatment site.

3. The surgical method of claim 2, wherein the at least one instrument driver includes a motor drive configured to move the working end helically.

4. The surgical method of claim 2, wherein the at least one instrument driver includes a motor drive configured to move the working end rotationally.

5. The surgical method of claim 2, wherein the at least one instrument driver includes a motor drive configured to move the working end axially.

6. The surgical method of claim 2, wherein the at least one instrument driver includes a piezoelectric actuator configured to jitter the first water jet stream.

7. The surgical method of claim 6, wherein the piezoelectric actuator jitters the first water jet stream at a rate of 1 Hz to 20 Hz.

8. The surgical method of claim 6, wherein the piezoelectric actuator jitters the first water jet stream in a stroke of at least 0.25 mm.

9. The surgical method of claim 6, wherein the piezoelectric actuator jitters the first water jet stream in a stroke ranging from 0.25 mm to 2.5 mm.

10. The surgical method of claim 6, wherein the piezoelectric actuator jitters the first water jet stream in a stroke ranging from 0.25 mm to 2.5 mm.

11. The surgical method of claim 2, wherein the at least one instrument driver includes a motor drive configured to jitter the first water jet stream.

12. The surgical method of claim 2, further comprising operating the controller, based on control logic, to operate the at least one instrument driver to control both movement and actuation of the first water jet stream to resect tissue in a mapped three-dimensional resection profile.

13. The surgical method of claim 11, wherein the treatment site is a prostate.

14.-37. (canceled)

38. A robotic medical method for resecting tissue, comprising:
introducing a working end of a resecting system into a prostate, wherein the working end carries an image sensor, a jetting orifice for jetting a first water jet stream that carries kinetic energy capable of resecting tissue and a second water vapor jet stream that undergoes a vapor-to-liquid phase change capable of cauterizing tissue;
propagating the first water jet stream thereby resecting tissue;
operating a controller to monitor video images from the image sensor and comparing artifacts in the video images with a library of resecting artifacts;
operating a controller, based on control logic, to adjust an operating parameter of the resecting system responsive to observing a resecting artifact.

39. The robotic medical method of claim 38, wherein the resecting artifact comprises at least one of an observable color that indicates bleeding, observable bubbles that indicate cavitation, observable walls of a resection cavity, observable tissue debris, observable color that indicating cauterization, observable verumontanum, observable ducts.

40. The robotic medical method of claim 38, wherein an operating parameter comprises at least one pressure of the first water jet stream, actuation of the first water jet stream, pulsation of the first water jet stream, a jitter rate of a piezoelectric actuator that moves the first water jet stream, a jitter stroke of the piezoelectric actuator, actuation of a second water jet stream; adjustment of cal/sec deliverable in the second water jet stream, movement of the working end, modulation of a negative pressure source communicating with the working end, and adjustment of a robot arm.

41. A surgical method for resecting a tissue prostate capsule to treat BPH, comprising:
introducing a mapping device in a trans-urethral approach into a prostate, and advancing at least one sensing needle with a dielectric sensor into prostate tissue to map the tissue prostate capsule in a plurality of locations; and
introducing a working end of a treatment device in a trans-urethral approach into the prostate, and delivering a first water jet stream and a second water jet stream int the prostate wherein the first water jet stream comprises a high-pressure liquid that carries kinetic energy thereby resecting prostate tissue and the second water jet stream comprises water vapor that undergoes a vapor-to-liquid phase change to cauterize prostate tissue;
wherein a controller, based on control logic, moves the working end and adjusts parameters of the first water jet stream corresponding to a map of the prostate produced by the mapping device.
